(19) United States
(12) Patent Application Publication
(10) Pub. No.: US 2026/0263832 A1
(43) Pub. Date: Sep. 10, 2026

(54) MULTI-CHANNEL INTRACAVITARY APPLICATOR FOR RADIATION ONCOLOGY

(71) Applicant: University of Kentucky Research Foundation, Lexington, KY (US)

(72) Inventors: Ali Soleimani-Meigooni, Lexington, KY (US); Ellis L. Johnson, Lexington, KY (US); Denise Fabian, Lexington, KY (US)

(21) Appl. No.: 19/559,331

(22) Filed: Mar. 6, 2026

Related U.S. Application Data
(60) Provisional application No. 63/768,084, filed on Mar. 6, 2025.
(60) Provisional application No. 63/768,295, filed on Mar. 7, 2025.

(51) Int. Cl. A61N 5/10; A61B 18/00; A61M 25/00

(52) U.S. Cl. CPC A61N 5/1002; A61B 2018/00559; A61M 25/003; A61N 2005/1005; A61N 2005/1021

(57) ABSTRACT

An intracavitary applicator for radiation oncology includes an elongated body that can be inserted into the vaginal canal of an individual. The elongated body includes a proximal end, a distal end, and a plurality of internal channels in which a radiation source can be inserted. The plurality of internal channels includes multiple channels that include a curved portion and are spaced from a longitudinal axis of the elongated body. The internal channels spaced from the longitudinal axis of the elongated body can be curved and the plurality of internal channels can be oriented in a manner that permits the elongated body to assume a reduced diameter compared to multi-channel vaginal applicators of known construction and/or facilitates reduced anisotropy during brachytherapy treatment.

### RELATED APPLICATIONS

This application claims priority to U.S. Patent Application Ser. No. 63/768,084 filed on Mar. 6, 2025 and U.S. Patent Application Ser. No. 63/768,295, filed on Mar. 7, 2025 the entire disclosure of each of which is incorporated herein by reference.

### TECHNICAL FIELD

The present disclosure generally relates to intracavitary applicators for radiation oncology. In particular, embodiments of the present disclosure relate to multi-channel vaginal applicators that make use of curved channels that do not converge toward a common point to reduce anisotropy of dose distribution during brachytherapy treatment and/or reduce applicator diameter.

### INTRODUCTION

Brachytherapy is a treatment used to treat various types of cancers, including vaginal, cervical, uterine, and endometrial cancer. During brachytherapy, a sealed radioactive source (or radiation source) is placed inside an applicator that is implanted in the body of the patient in proximity to a tumor or an area from which a tumor was excised. Intracavitary placement of the radiation source during brachytherapy generally occurs around a natural body cavity. This technique facilitates a more targeted and concentrated dose of radiation to the cancer cells while minimizing damage to surrounding healthy tissues relative to the alternative method of treatment with external beam radiation. Brachytherapy can be utilized alone or in combination with other cancer treatment techniques, such as surgery, external beam radiotherapy and chemotherapy. Based on the location and size of the tumor, different types of applicators have been designed and gradually modified to improve quality treatments.

Patients with uterine cancer or cervical cancer sometimes receive a hysterectomy as part of their treatment. In order to prevent recurrence at the surgical site, some patients undergo vaginal cuff brachytherapy in which a vaginal cylinder applicator adapted to position the radiation source into the vagina of the patient delivers the required radiation to the vaginal cuff of the patient. Presently available vaginal cylinder applicators utilized for such treatment typically include a single channel at the center of the applicator in which the radiation source is placed, as shown in FIG. 1. This single-channel construction is, however, limiting with respect to the radiation dose to the actual target due to what is known as anisotropy (non-uniformity of radiation from the source due to the source structure), as further shown in FIG. 1. As a result, treatment volume does not result in a homogenous dose of radiation. In this regard, there is an under-dosing of radiation at the apex of such applicators, where treatment volume is located. It is critical that the vaginal cuff is dosed properly as it is the main target and site of recurrence for cancer following hysterectomy. Although a multi-channel vaginal cylinder applicator has been developed in an effort to solve this issue (see FIGS. 2 and 3), the design of such applicator created a size/diameter that is too large for many cancer patients, thus rendering the applicator impractical for use.

### SUMMARY

This summary describes several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

The present disclosure includes intracavitary applicators useful in radiation oncology applications, such as brachytherapy treatment. In particular, the present disclosure includes intracavitary applicators in the form of multi-channel vaginal applicators that make use of curved channels to reduce dose distribution during brachytherapy treatment and/or applicator diameter.

An exemplary intracavitary applicator for radiation oncology includes an elongated body that can be inserted into a vaginal canal of an individual. The elongated body includes a proximal end, a distal end, and a plurality of internal channels in which a radiation source can be inserted and guide the radiation source from the proximal end toward the distal end of the elongated body. The plurality of internal channels includes multiple internal channels that are spaced from a longitudinal axis of the elongated body. The internal channels spaced from the longitudinal axis of the elongated body can also be characterized as “outer channels.” The outer channels include a curved portion. The curved portion of the outer channels can have curvature characteristics that assist in reducing anisotropy during brachytherapy treatment. The outer channels can be curved and the plurality of internal channels of the elongated body oriented so that the plurality of internal channels do not converge toward a common point in the elongated body to enable the elongated body to assume a reduced diameter compared to multi-channel vaginal applicators of known construction. In some embodiments, the plurality of internal channels includes a central channel that extends along the longitudinal axis of the longitudinal body and to which the outer channels are spaced from.

In some embodiments, the curved portion of each of the outer channels of the elongated body curves toward another adjacently positioned outer channel. In some embodiments, the curvature of the curved portion of each of the outer channels reduces the distance between the outer channel and the longitudinal axis of the longitudinal body. In some embodiments, the curved portion of one or more of the outer channels curves in multiple directions. In some embodiments, the curved portion of each outer channel is such that the outer channel cannot be bisected by a single vertical plane. In some embodiments, each internal channel of the plurality of internal channels can be bisected by a single vertical plane.

In some embodiments, the elongated body includes a cylindrical portion that defines the proximal end of the elongated body and a dome portion that defines the distal end of the elongated body. In some embodiments, each internal channel of the plurality of internal channels extends from the proximal end of the elongated body to a terminal point located in the dome portion of the elongated body.

Various dimensions of the elongated body can be adjusted to accommodate different individual anatomies and/or brachytherapy applications. In some embodiments, the elongated body has an outer diameter that is less than 3.5 centimeters (cm). In some embodiments, the elongated body has an outer diameter ranging from about 2.5 cm to about 3.5 cm.

In some embodiments, the intracavitary applicator further includes a plurality of conduits attached to the elongated body. Each conduit of the plurality of conduits can be attached as to be in fluid communication with, and can facilitate the transfer of a radiation source to, an internal channel of the plurality of internal channels. In some embodiments, the intracavitary applicator has a length of about 25 cm.

### BRIEF DESCRIPTION OF THE DRAWINGS

FIG. 1 is a computed tomography (CT) image of a prior art single-channel vaginal cylinder applicator inserted in the vaginal cavity of an individual and labeled with treatment point and 100% isodose indicator lines;

FIG. 2 is a prior art multi-channel vaginal cylinder applicator (known as a Wright applicator);

FIG. 3 is a CT image showing the internal channels of the prior art multi-channel vaginal cylinder applicator of FIG. 2 converging toward a common point;

FIG. 4 is a perspective view an intracavitary applicator made in accordance with an embodiment of the present disclosure;

FIG. 5 is another perspective view of the intracavitary applicator of FIG. 4;

FIG. 6 is a side view of the intracavitary applicator of FIG. 4, with a plurality of conduits attached to an elongated body of the intracavitary applicator;

FIG. 7 is a cross-sectional view of the intracavitary applicator taken along 7-7 in FIG. 4;

FIG. 8 is a cross-sectional view of the intracavitary applicator taken along 8-8 in FIG. 4;

FIG. 9 is a cross-sectional view of the intracavitary applicator taken along 9-9 in FIG. 4, providing an opposite view of that of FIG. 8;

FIG. 10 is a perspective view of the portions of the elongated body that define internal channels of the intracavitary applicator of FIG. 4;

FIG. 11 is a schematic representation of the intracavitary applicator of FIG. 4 deposited in a vaginal cavity and proximal to a vaginal cuff of an individual following a total hysterectomy;

FIG. 12 is a perspective view of an intracavitary applicator made in accordance with another embodiment of the present disclosure;

FIG. 13 is a cross-sectional view of the intracavitary applicator taken along 13-13 in FIG. 12;

FIG. 14 is a cross-sectional view of the intracavitary applicator taken along 14-14 in FIG. 12;

FIG. 15 is a cross-sectional view of the intracavitary applicator taken along 15-15 in FIG. 12;

FIG. 16 is a perspective view of an intracavitary applicator made in accordance with another embodiment of the present disclosure;

FIG. 17 is a cross-sectional view of the intracavitary applicator taken along 17-17 in FIG. 16;

FIG. 18 is a perspective view of the portions of an elongated body that define internal channels of the intracavitary applicator of FIG. 16;

FIG. 19 is a top view of the portions of the elongated body that define the internal channels of the intracavitary applicator of FIG. 16.

### DESCRIPTION OF EXEMPLARY EMBODIMENTS

The details of one or more embodiments of the presently disclosed subject matter are set forth in this document. Modifications to embodiments described in this document, and other embodiments, will be evident to those of ordinary skill in the art after a study of the information provided in this document. The information provided in this document, and particularly, the specific details of the described exemplary embodiments, is provided primarily for clearness of understanding and no unnecessary limitations are to be understood therefrom. In case of conflict, the specification of this document, including definitions, will control.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Unless otherwise indicated, all numbers expressing quantities of dimension and angle used in the specification are to be understood as being modified in all instances by the term “about”. Accordingly, unless indicated to the contrary, the numerical parameters set forth in this specification are approximations that can vary depending upon the desired properties sought to be obtained by the presently disclosed subject matter.

As used herein, the term “about,” when referring to a value or to dimension or angle is meant to encompass variations of in some embodiments±20%, in some embodiments±10%, in some embodiments±5%, in some embodiments±1%, in some embodiments±0.5%, in some embodiments±0.1%, in some embodiments±0.01%, and in some embodiments±0.001% from the specified amount, as such variations are appropriate to perform the disclosed method.

It is understood that where a range of units is disclosed, such range is inclusive of the starting and end units. For example if a range of “10 to 15” or “between 10 to 15” is disclosed, then 10 and 15 are considered part of such range, unless stated otherwise. It is also understood that each unit between two particular units are also disclosed. For example, if 10 and 15 are disclosed, then 11, 12, 13, and 14 are also disclosed.

The present disclosure includes intracavitary applicators. In particular, certain embodiments of the present disclosure are directed to multi-channel vaginal applicators that make use of curved channels that do not converge toward a common point to reduce applicator diameter and/or anisotropy during brachytherapy treatment of an individual, such as a human born biologically female. Cancers for which brachytherapy treatment can be applied as treatment include, by way of non-limiting example, vaginal, cervical, uterine, and endometrial cancer. As will be appreciated by one of ordinary skill in the art, upon study of this document, intracavitary applicator embodiments disclosed herein may be utilized to provide more homogenous radiation dosing and/or reduce discomfort during brachytherapy treatment.

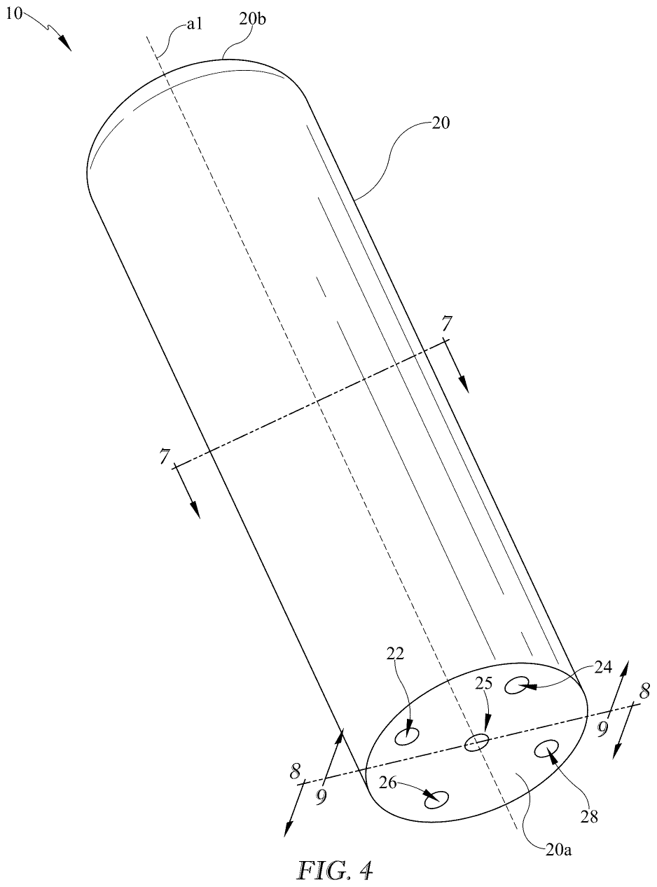

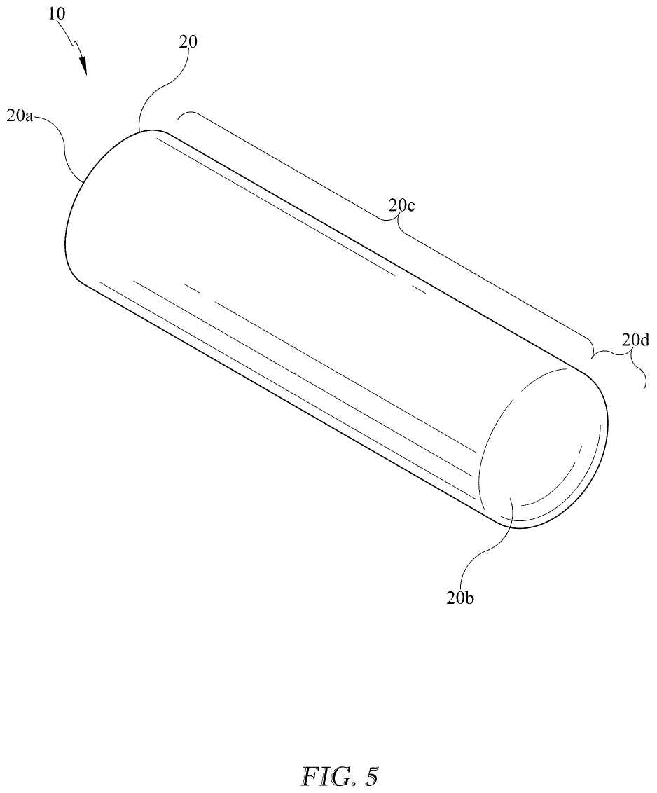

Referring first to FIGS. 4-6, an exemplary intracavitary applicator in the form of a multi-channel vaginal applicator (or applicator) 10 is provided and includes: an elongated body 20 that is configured to be inserted into the vaginal canal of an individual. The elongated body 20 includes a proximal end 20a, a distal end 20b, and a plurality of internal channels 22, 24, 25, 26, 28 with an open proximal end and a closed distal end. Each internal channel 22, 24, 25, 26, 28 is configured to receive a radiation source (not shown) and provides a pathway along which the received radiation source can travel from the proximal end 20a toward the distal end 20b of the elongated body 20.

In use, the applicator 10 can be utilized to apply brachytherapy treatment of cervical and/or uterine cancer by depositing the applicator 10 into the vaginal canal of an individual so that the distal end 20b of the elongated body 20 is near the vaginal cuff and/or cervix. Once the applicator 10 is deposited, a radiation source can be sequentially inserted into the internal channels 22, 24, 25, 26, 28 to apply radiation dosing to a target treatment area.

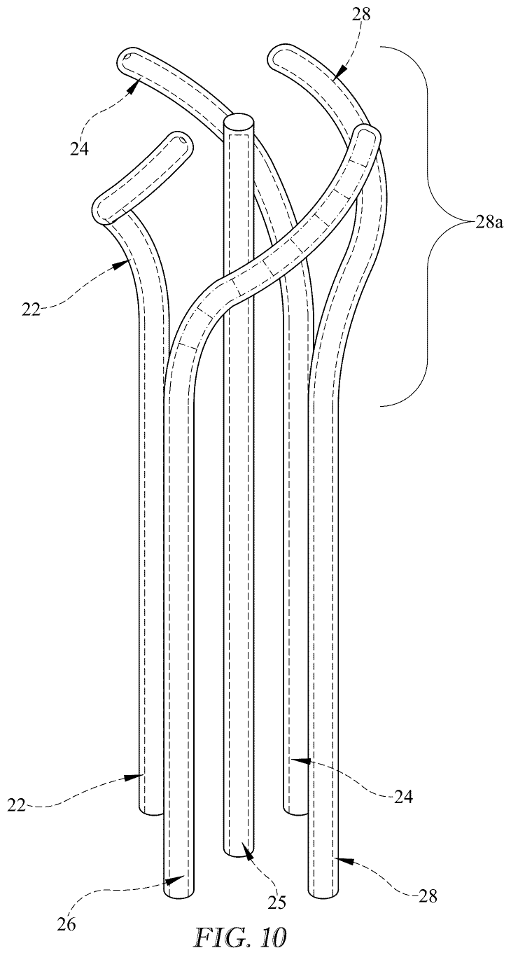

The radiation source can be moved to different locations along the internal channels 22, 24, 25, 26, 28 to apply radiation dosing to different areas of the individual's anatomy. In this regard, during brachytherapy, such as high-dose radiation (HDR) brachytherapy, the radiation source can be attached to the end of a wire and can be selectively deposited in the internal channels 22, 24, 25, 26, 28 at different positions therein to facilitate treatment of an individual. Treatment planning software can be utilized to determine the positions in the respective channels 22, 24, 25, 26, 28 that the radiation source will stop for a given period of time to deliver the required radiation prior to moving to a different location in the channel 22, 24, 25, 26, 28 for a subsequent period of time at such location, and so on. These positions are known as “dwell” positions and the time that the radiation source remains in that position is known as “dwell” time. The radiation source and/or dwell positions along each channel 22, 24, 25, 26, 28 while the applicator 10 is in use can be determined based on the known length of the radiation source. In some embodiments, each respective channel 22, 24, 25, 26, 28 is configured to accommodate multiple dwell positions per centimeter of the treatment length (i.e., length of a selected section of the patient's anatomy targeted for treatment). For instance, in some embodiments, the radiation source may have a length of 5 millimeters (mm) and each channel 22, 24, 25, 26, 28 may be configured to accommodate two dwell positions per centimeter of the treatment length. Accordingly, if in such embodiments, a physician or radiation oncologist contour the treatment segment of a computed tomography (CT) scan to have a 4 cm length, each respective channel 22, 24, 25, 26, 28 would be able to accommodate eight dwell positions. In FIG. 10, eight example dwell positions for a radiation source are indicated in rectangular, dot-dash lines.

Referring now to FIGS. 4-9, the distal end 20b of the elongated body 20 is rounded to facilitate insertion of the applicator 10 in to the vaginal canal of an individual. Specifically, in this embodiment, the elongated body 20 is shaped, such that the elongated body 20 can be characterized as including a cylindrical portion 20c and a dome portion 20d. The cylindrical portion 20c defines the proximal end 20a of the elongated body 20 and extends from a proximal end 20a of the elongated body 20 to the dome portion 20d, which defines the distal end 20b of the elongated body 20. In this embodiment, each internal channel 22, 24, 25, 26, 28 extends from the proximal end 20a of the elongated body 20 to a terminal point that is located in the dome portion 20d of the elongated body 20, as best shown in FIGS. 4 and 7-9. Accordingly, in this embodiment, the proximal end of the cylindrical portion 20c defines the open, proximal end of each internal channel 22, 24, 25, 26, 28 in which a radiation source is initially received into the elongated body 20, and the dome portion 20d defines the closed distal end of each internal channel 22, 24, 25, 26, 28 that limits the extent to which the radiation source can travel through the elongated body 20. Alternative embodiments, in which some or all of the internal channels 22, 24, 25, 26, 28 terminate at a point that is located at a distal portion of the cylindrical portion 20c of the elongated body 20 are, however, also contemplated herein. In this embodiment, the cylindrical portion 20c and the dome portion 20d are integrally formed, such that the cylindrical portion 20c and the dome portion 20d is formed by a unitary member. Alternative embodiments in which the cylindrical portion 20c and the dome portion 20d are separate members joined together are, however, also contemplated herein.

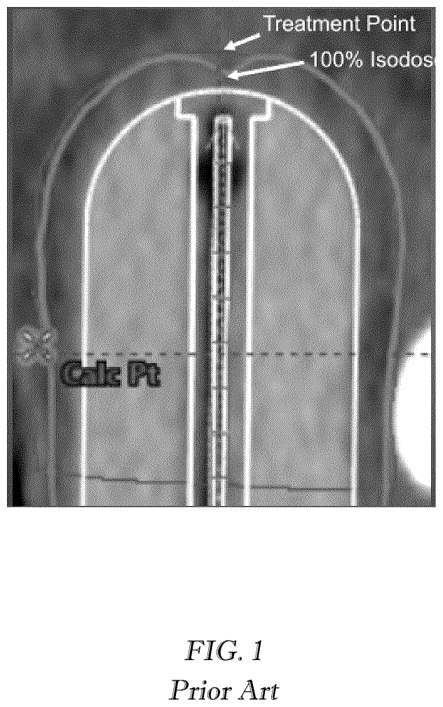

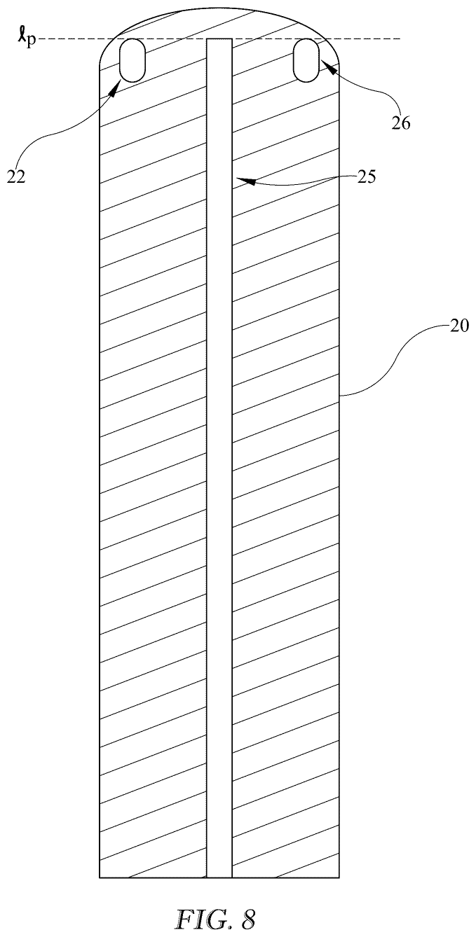

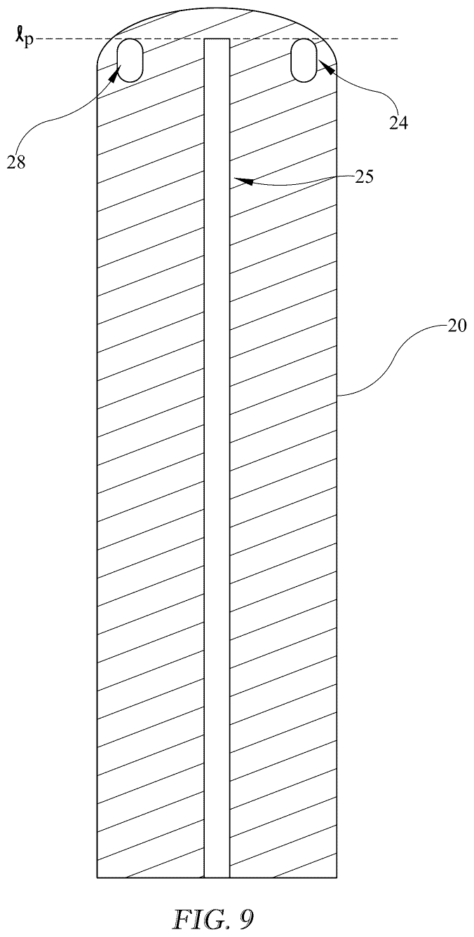

Referring now to FIGS. 1, 7, and 8-11, the elongated body 20 includes multiple internal channels 22, 24, 26, 28 that are spaced from a longitudinal axis, a₁, of the elongated body 20. Specifically, in this embodiment, there are: four internal channels 22, 24, 26, 28 that are spaced from the longitudinal axis, a₁, of the elongated body 20; and one internal channel 25 that extends along the longitudinal axis, a₁, of the elongated body 20. Accordingly, in this embodiment, the elongated body 20 defines a total of five internal channels 22, 24, 25, 26, 28 in which a radiation source can be received. As the internal channel 25 extending along the longitudinal axis, a₁, is centrally positioned in this embodiment, internal channel 25 can also be characterized as a “central channel”. As the four internal channels 22, 24, 26, 28 are spaced from the longitudinal axis, a₁, and thus the central channel 25, these four internal channels can also be characterized as “outer channels”. As best shown in FIGS. 8 and 9, in this embodiment, at least a portion of the terminus of each respective internal channel 22, 24, 25, 26, 28 is located along a common lateral plane, lₚ, extending transverse to the longitudinal axis.

Referring now to FIGS. 4 and 7-9, in this embodiment, the internal channels 22, 24, 25, 26, 28 are spaced apart from each other, such that the central channel 25 is generally equidistant to each respective outer channel 22, 24, 26, 28. In various embodiments, select portions or the entirety of each outer channel 22, 24, 26, 28 can be equidistantly spaced from the central channel 25. As shown best in FIG. 8, select portions of the entirety of each outer channel 22, 24, 26, 28 can be equidistantly spaced from two other outer channels 22, 24, 26, 28, and, in particular, the two other outer channels 22, 24, 26, 28 to which it is adjacently positioned. Without wishing to be bound by any particular theory, the equidistant positioning of the central channel 25 relative to the outer channels 22, 24, 26,28 and equidistant positioning of each outer channel 22, 24, 26, 28 relative to the two outer channels 22, 24, 26, 28 to which its adjacently positioned is believed to help facilitate uniform radiation dosing when the elongated body 20 is loaded with a radiation source and the applicator 10 is in use.

Referring now to FIG. 10, each outer channel 22, 24, 26, 28 includes a curved portion 28a that defines the distal end of the outer channel 22, 24, 26, 28, and the central channel 25 is linear. The curved portion 28a of the outer channels 22, 24, 26, 28 are provided toward the distal end 20b of the elongated body 20, where the treatment volume will be proximally located when the applicator 10 is in use. The curvature of the outer channels 22, 24, 26, 28 serve to divert the anisotropy portion of the radiation source in each outer channel 22, 24, 26, 28 away from the treatment volume and thus improve treatment dose. For clarity in illustration, only the curved portion 28a of outer channel 28 is provided with a reference numeral. It is appreciated, however, that each outer channel 22, 24, 26, 28 includes a curved portion despite not being provided with an identifying reference numeral. In some embodiments, the curvature exhibited by each outer channel 22, 24, 26, 28 is identical. In this embodiment, the curved portion 28a of each of the outer channels 22, 24, 26, 28 curves in multiple directions. Specifically, in this embodiment, the curved portion 28a of each of the outer channels 22, 24, 26, 28 curves in multiple directions, such that none of the outer channels 22, 24, 26, 28 can be bisected by a single vertical plane. In this embodiment, the curvature exhibited by the curved portion 28a of each outer channel 22, 24, 26, 28 is also such that curved portion 28a of the outer channel 22, 24, 26, 28 extends upwardly and toward another, adjacently positioned outer channel 22, 24, 26, 28, without any two of the outer channels 22, 24, 26, 28 curving toward each other. Specifically, in the embodiment shown in FIG. 10, the curved portion 28a of each outer channel 22, 24, 26, 28 assumes a semi-helical shape, where: the curved portion of outer channel 26 extends upwardly and toward outer channel 28; the curved portion 28a of outer channel 28 extends upwardly and toward outer channel 24; the curved portion of outer channel 24 extends upwardly and toward outer channel 22; and the curved portion of outer channel 22 extends upwardly and toward outer channel 26.

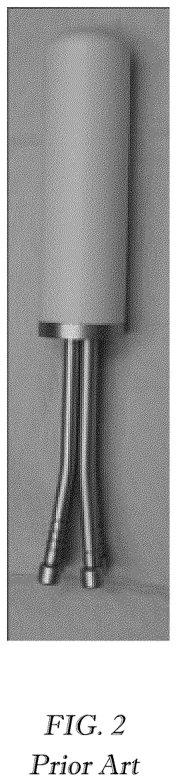

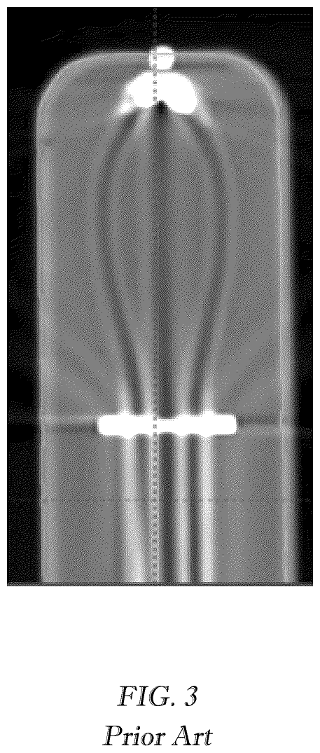

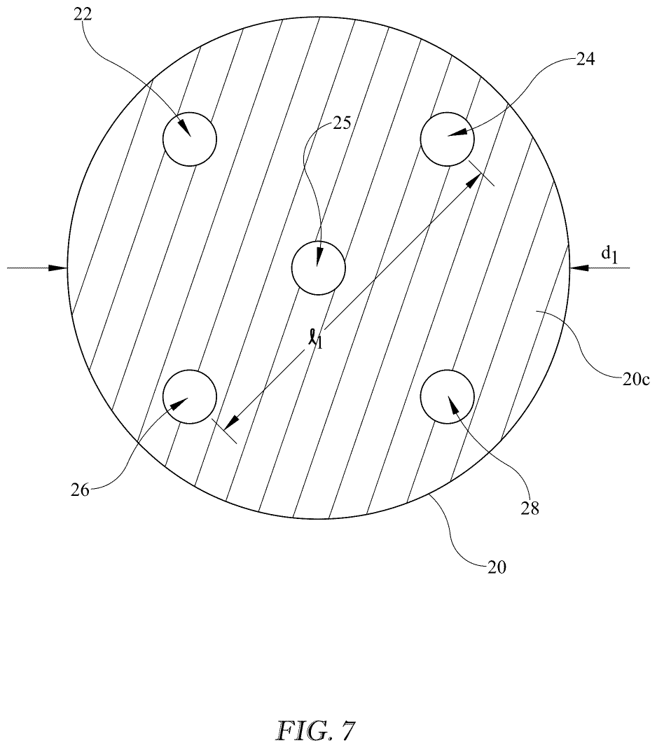

Referring now to FIGS. 4 and 7-11, in this embodiment, no two outer channels 22, 24, 26, 28 curve toward each other, and the internal channels 22, 24, 25, 26, 28 do not converge toward or meet at a common point in the elongated body 20. As a result, the closed distal end of each internal channel 22, 24, 25, 26, 28 occupies a discrete position within the elongated body 20 that is spaced apart from the closed distal end of the other internal channels 22, 24, 25, 26, 28. Thus, unlike multi-channel applicators of known construction in which the channels do converge toward or meet at a common point, like that shown in FIGS. 2 and 3, the internal channels 22, 24, 25, 26, 28 of the applicator 10 do not collectively assume a bulbous shape that may necessitate a larger applicator diameter. The orientation of the internal channels 22, 24, 25, 26, 28 and curvature of the outer channels 22, 24, 25, 26, 28 of the applicator 10 thus permit greater flexibility of the spacing between the internal channels 22, 24, 25, 26, 28, which, in turn, permits greater flexibility with respect to the sizing of the diameter, d₁, (FIG. 7) of the elongated body 20. As such, the elongated body 20 can assume a smaller diameter relative to that of multi-channel applicators of known construction and reduce discomfort during insertion of the applicator 10 into, or while the applicator 10 is deposited in, the vaginal canal of an individual during brachytherapy treatment.

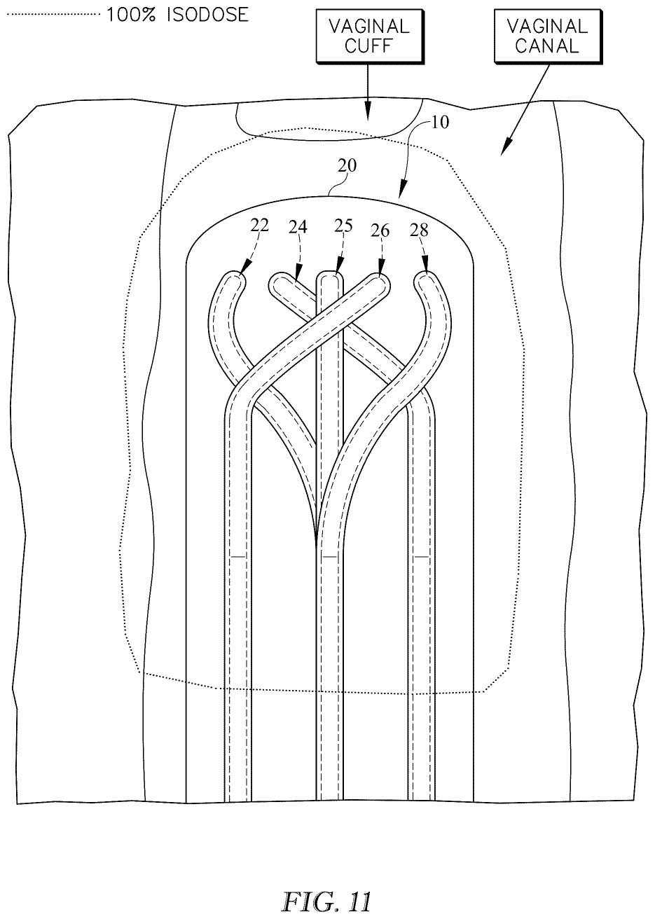

Referring now specifically to FIG. 11, and without wishing to be bound by theory, the orientation of the internal channels 22, 24, 25, 26 and curvature exhibited by the outer channels 22, 24, 26, 28 is believed facilitate the positioning of a radiation source throughout the elongated body 20 in a manner that reduces anisotropy during brachytherapy treatment employing the applicator 10. The reduction in anisotropy can, in turn, reduce underdosing and improve the homogeneity of dosing provided at the distal end 20b of the elongated body 20. Accordingly, when utilized for vaginal cuff brachytherapy, the applicator 10 may serve to provide more uniform radiation distribution at the target treatment site than single-channel vaginal cylinder applicators of known construction, as evidenced by comparing the isodose lines and treatment points in FIGS. 1 and 11, and multi-channel vaginal cylinder applicators employing linear channels arranged in parallel. In this regard, it is noted that in standard vaginal cylinder applicators with a single channel, the radiation source is typically oriented in a manner where anisotropy of the radiation source undesirably occurs at the target treatment volume of the subject. As noted, the curvature of the outer channels 22, 24, 26, 28 serves to divert the orientation of the radiation source and the anisotropy thereof away from the target treatment volume.

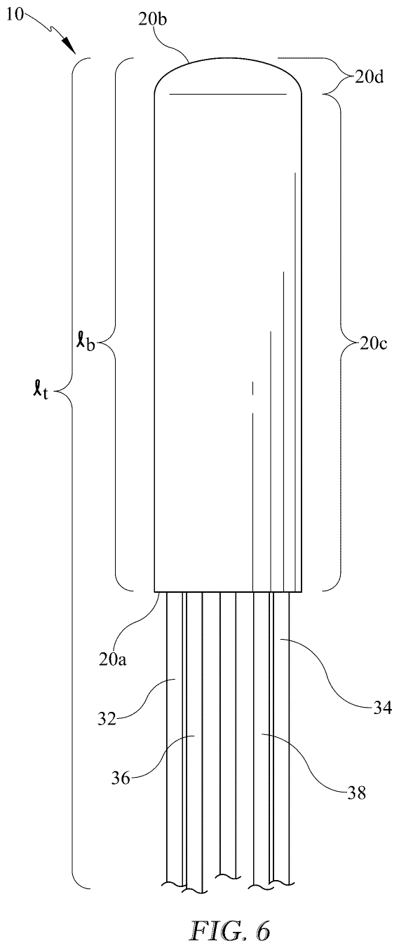

Referring now specifically to FIG. 6, in some embodiments, the applicator 10 can further include a plurality of conduits 32, 34, 35, 36, 38 that are configured to receive and guide a radiation source into the internal channels 22, 24, 25, 26, 28 of the elongated body 20. In this regard, each respective conduit 32, 34, 35, 36, 38 is in fluid communication with an internal channel 22, 24, 25, 26, 28 of the elongated body 20. The conduits 32, 34, 35, 36, 38 may also be characterized as source transfer tubes.

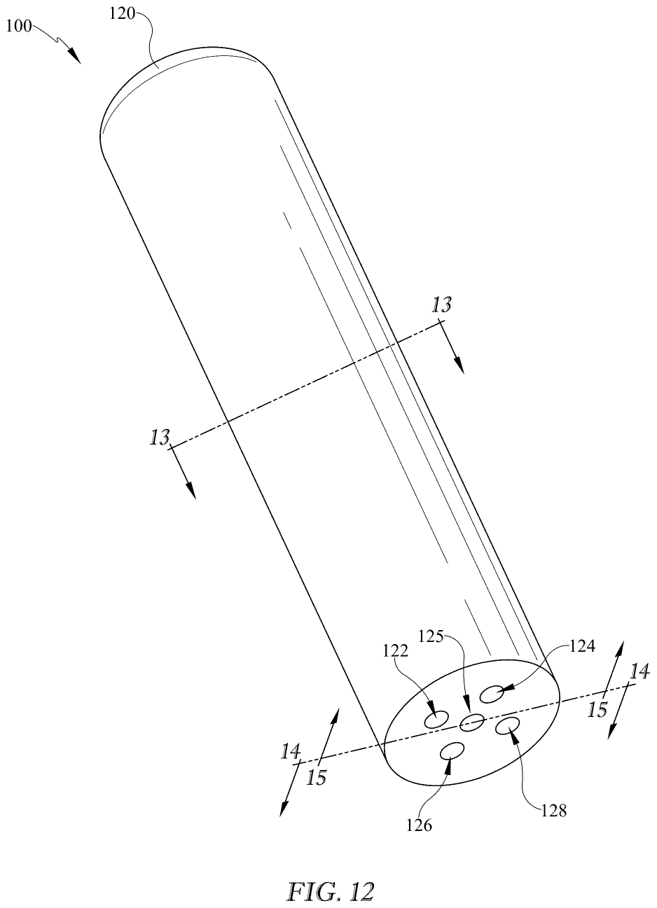

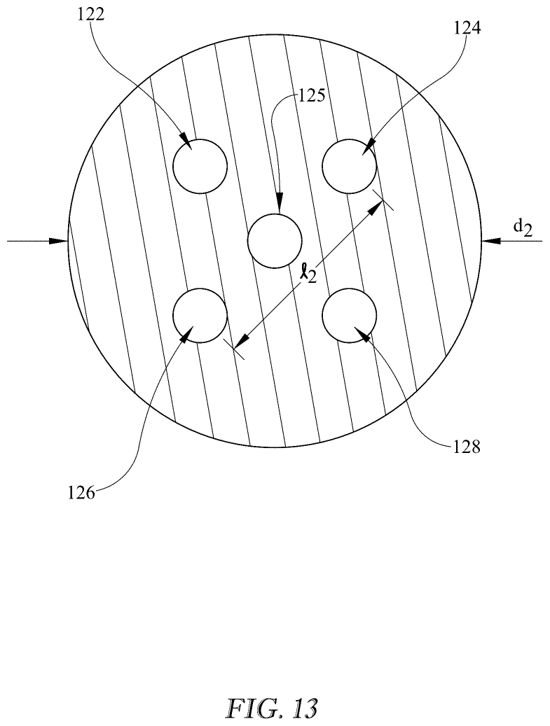

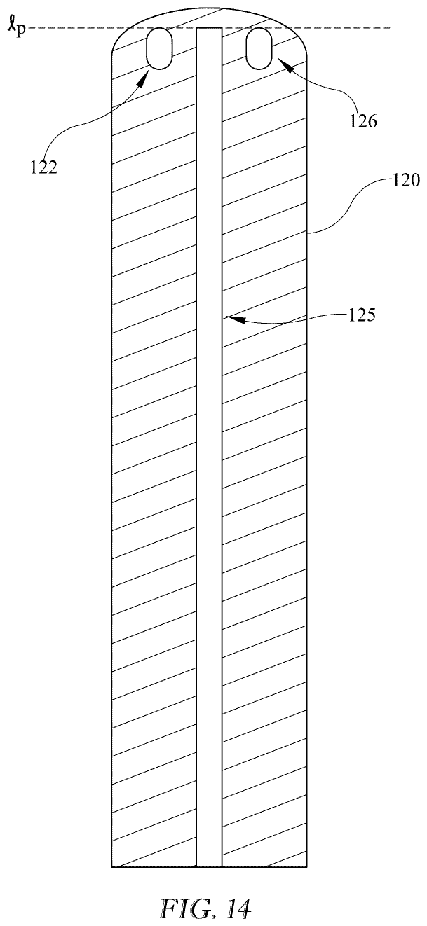

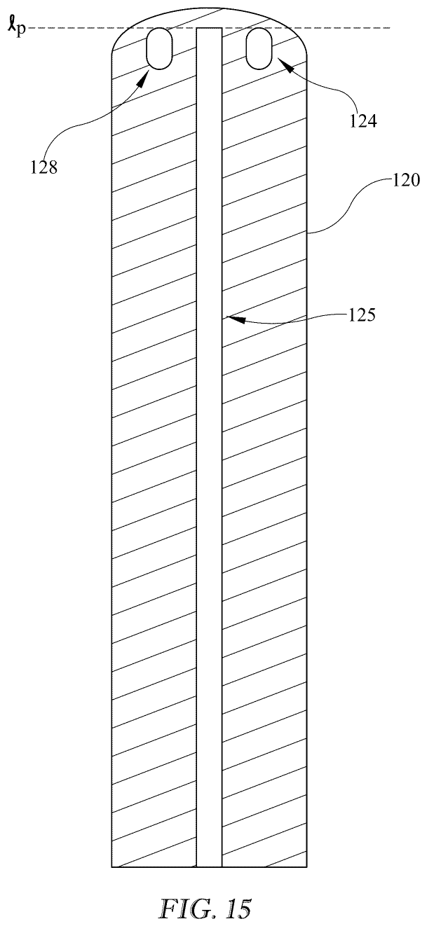

Referring now to FIGS. 12-15, another exemplary embodiment of an intracavitary applicator in the form of a multi-channel vaginal applicator (or applicator) 100 is provided. The applicator 100 in this embodiment is of the same general construction and functions in the same manner as the applicator 10 described above with reference to FIGS. 4-11, except that the spacing of the outer channels 122, 124, 126, 128 relative to both each other and to the central channel 125 is reduced. Such reduced spacing between, and the non-converging nature of, the internal channels 122, 124, 125, 126, 128 enables further reduction in the diameter of the elongated body 120, which may prove particularly advantageous during brachytherapy treatment of individuals with narrower vaginal cavities.

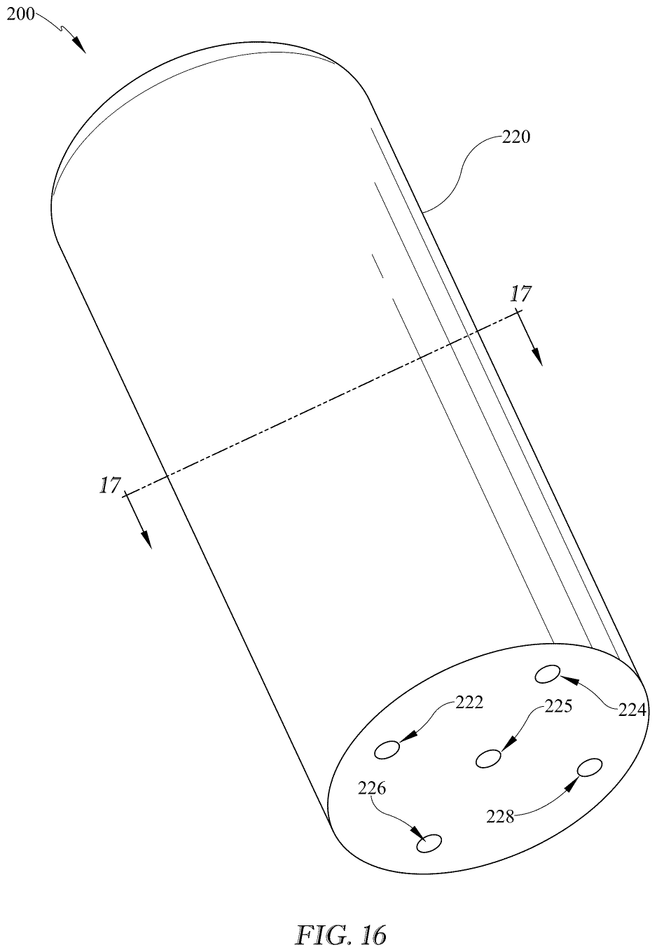

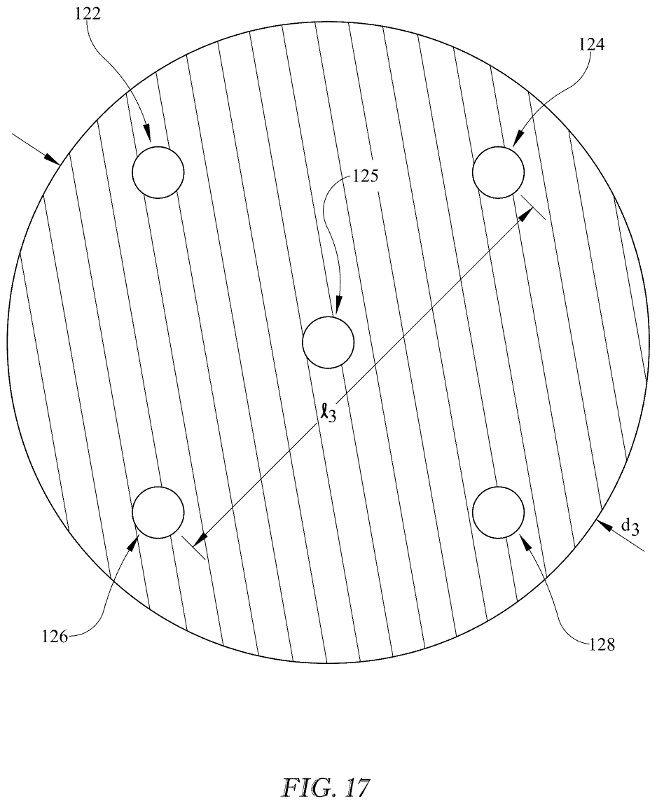

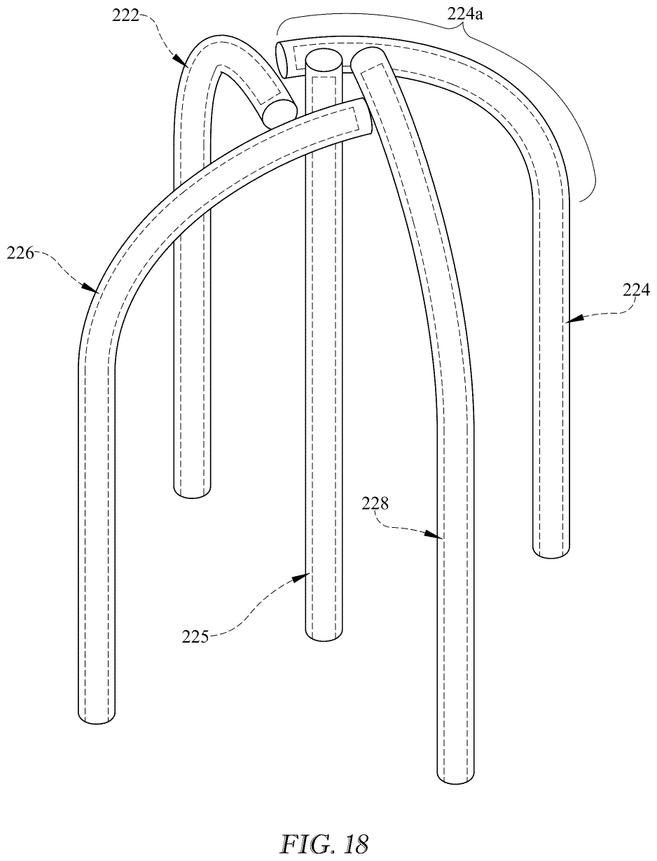

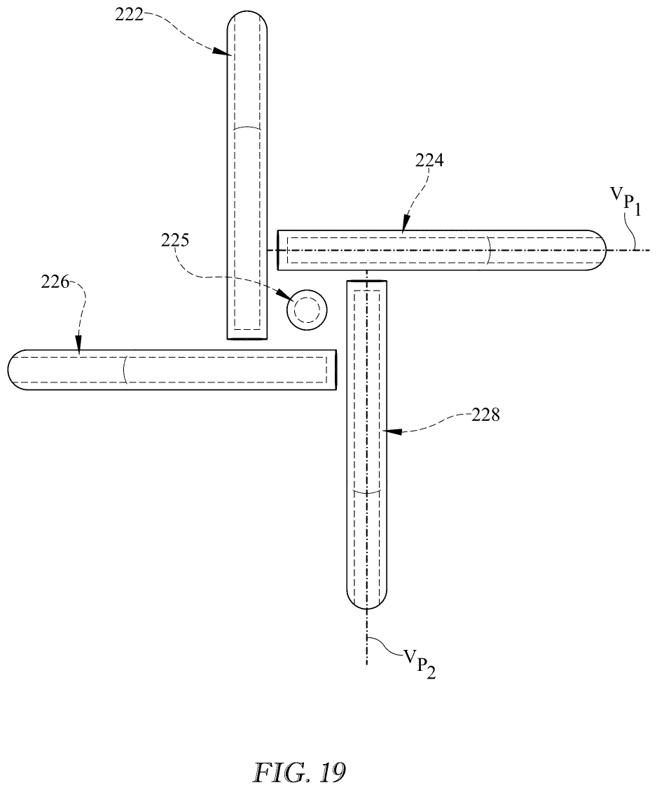

Referring now to FIGS. 16-19, another exemplary embodiment of an intracavitary applicator in the form of a multi-channel vaginal applicator (or applicator) 200 is provided. The applicator in this embodiment is of the same general construction and functions in the same manner as the applicators 10, 100 described above with reference to FIGS. 4-15. However, in this embodiment, the outer channels 222, 224, 226, 228 are spaced further apart from both each other and the central channel 225 and exhibit different curvature characteristics than the outer channels 22, 24, 26, 28, 122, 124, 126, 128 of the applicators 10, 100 described above with reference to FIGS. 4-15. In this embodiment, the curved portion 224a of each outer channel 222, 224, 226, 228 curves in a single direction. Specifically, in this embodiment, the curved portion 224a of each outer channel 222, 224, 226, 228 curves in a single direction, such that each outer channel 222, 224, 226, 228 can be bisected by a single vertical plane, as reflected by the vertical plane, vₚ₁, bisecting outer channel 224 and the vertical plane, vₚ₂, bisecting outer channel 228 in FIG. 19. The linear central channel 225 can also be bisected by a single vertical plane. In this embodiment, the curved portion 224a of each outer channel 222, 224, 226, 228 causes the spacing between the distal end of each outer channel 222, 224, 226, 228 and the central channel 225 to be reduced relative to the spacing between the proximal end of each outer channel 222, 224, 226, 228 and the central channel 225, as shown best in FIGS. 18 and 19. For clarity in illustration, only the curved portion 224a of outer channel 224 is provided with a reference numeral. It is appreciated, however, that each outer channel 222, 224, 226, 228 includes a curved portion.

Referring now specifically to FIGS. 18 and 19, the curvature exhibited by the curved portion 224a of each outer channel 222, 224, 226, 228 is such that the curved portion 224a of the outer channel 222, 224, 226, 228 curves toward another, adjacently positioned outer channel 222, 224, 226, 228, without any two of the outer channels 222, 224, 226, 228 curving toward each other, and without the internal channels 222, 224, 226, 228 converging toward or meeting at a common point in the elongated body 220. Specifically, in this embodiment, the curved portions 224a of the outer channels 222, 224, 226, 228 are oriented such that the vertical plane, vₚ₁, vₚ₂, bisecting each outer channel 222, 224, 226, 228 is perpendicular to the vertical plane, vₚ₁, vₚ₂, of the adjacently positioned outer channel 222, 224, 226, 228 that it curves toward, as best shown in FIG. 19. As shown in FIG. 18, in this embodiment, each outer channel 222, 224, 226, 228 retains partial arch-like shape, while the central channel 225 is linear. Thus, in this embodiment, the lateral area of the outer channels 222, 224, 226, 228 proximate to the distal end of the elongated body 220 is increased relative to that of the applicators 10, 100 described above with reference to FIGS. 4-15. The curvature of the outer channels 222, 224, 226, 228 divert the anisotropy portion of the radiation source in each outer channel 222, 224, 226, 228 away from the treatment volume and thus improves treatment dose.

Applicators with various diameters and channel spacings can be constructed in accordance with the present disclosure to accommodate the anatomies of a wide range of individuals. For instance, in some embodiments and implementations, the elongated body 120 of the applicator 100 described above with reference to FIGS. 12-15 can have a diameter, d₂, of about 2.5 centimeters (cm) and have an opposing outer channel 122, 124, 126, 128 distance, l₂, of about 1.5 cm to facilitate brachytherapy treatment for individuals with narrower vaginal cavities, smaller cervixes, and/or smaller vaginal cuffs. In some embodiments and implementations, the elongated body 220 of the applicator 200 described above with reference to FIGS. 16-19 can have a diameter, d₃, of about 3.5 cm and have an opposing outer channel 222, 224, 226, 228 distance, l₃, of about 2.5 cm to facilitate brachytherapy treatment for individuals with larger vaginal cavities, cervixes, and/or vaginal cuffs. In some embodiments and implementations, the elongated body 20 of the applicator 10 described above with reference to FIGS. 4-11 can have a diameter, d₁, of about 3.0 cm and have an opposing outer channel 22, 24, 26, 28 distance, l₁, of about 2.0 cm to facilitate brachytherapy treatment for individuals with average sized vaginal cavities, cervixes, and/or vaginal cuffs. It should be appreciated, however, that the disclosed applicators 10, 100, 200 are not restricted to the specific diameters, outer channel spacings, and diameter-outer channel spacing combinations indicated in the foregoing example embodiments. In various embodiments, the elongated body 20, 120, 220 of the respective applicators can have a diameter, d₁, d₂, d₃ ranging from about 2.0 cm to about 3.5 cm and an opposing outer channel distance, l₁, l₂, l₃, ranging from about 1.5 cm to about 2.5 cm.

Applicators with various lengths can also be constructed in accordance with the present disclosure to accommodate the anatomies of a wide range of individuals. For instance, in some embodiments, the elongated body 20, 120, 220 of the applicator 10, 100, 200 can have a length, l_{b}, ranging from about 6 cm to about 10 cm. In some embodiments where the applicator 10, 100, 200 further includes conduits 32, 34, 35, 36, 38 for receiving and guiding a radiation source into the internal channels 22, 24, 25, 26, 28, 122, 124, 125, 126, 128, 222, 224, 225, 226, 228, the total length, lₜ, of the applicator 10, 100, 200 can be about 25 cm. Although the conduits 32, 34, 35, 36, 38 are only illustrated in the drawings and described above in connection with applicator 10, it is appreciated that applicator 100 and applicator 200 described above can, in some embodiments, also include such conduits 32, 34, 35, 36, 38.

As reflected in TABLE 1 below, various embodiments of applicators 10, 100, 200 can be established by modifying: the diameter of the elongated body 20, 120, 220; opposing outer channel 22, 24, 26, 28, 122, 124, 126, 128, 222, 224, 226, 228 distance, l₁, l₂, l₃; and/or length, l_{b} of the elongated body 20, 122, 124. Various embodiments can also be established by using adding, removing, or using conduits 32, 34, 35, 36, 38 of different lengths.

TABLE 1

| | | Opposing | | |
|---|---|---|---|---|
| | Body | Outer | | Total Length |
| | Diameter | Channel | Length of | Including |
| Applicator | (d₁, d₂, d₃) | Distance (l₁, | Body (l_{b}) | Conduits |
| # | (cm) | l₂, l₃) (cm) | (cm) | (lₜ) (cm) |
| 1 | 2.5 | 1.5 | 6 | 25 |
| 2 | 2.5 | 1.5 | 7 | 25 |
| 3 | 2.5 | 1.5 | 8 | 25 |
| 4 | 2.5 | 1.5 | 9 | 25 |
| 5 | 2.5 | 1.5 | 10 | 25 |
| 6 | 3 | 2 | 6 | 25 |
| 7 | 3 | 2 | 7 | 25 |
| 8 | 3 | 2 | 8 | 25 |
| 9 | 3 | 2 | 9 | 25 |
| 10 | 3 | 2 | 10 | 25 |
| 11 | 3.5 | 2.5 | 6 | 25 |
| 12 | 3.5 | 2.5 | 7 | 25 |
| 13 | 3.5 | 2.5 | 8 | 25 |
| 14 | 3.5 | 2.5 | 9 | 25 |
| 15 | 3.5 | 2.5 | 10 | 25 |

It is appreciated that while the applicators 10, 100, 200 described above with reference to FIGS. 4-19 are primarily referred to as including five internal channels that, in alternative embodiments, additional or fewer internal channels may be utilized and that the curvature exhibited by the outer channels in such embodiments can be adjusted to provide desired dosing at a target treatment area when the applicators 10, 100, 200 are in use. For instance, in some embodiments the number of internal channels, positioning of the internal channels, and/or shape of the elongated body of the applicator 10, 100, 200 may be adjusted to further accommodate brachytherapy treatment for tumors that are not symmetric in an individual's anatomy and require more radiation on one side of the individual than the other side of the individual.

The length of the non-curved portion and/or curved portion of the internal channels can also be adjusted to accommodate different anatomies and/or brachytherapy applications. The number of directions in which the curved portion of the internal channels curve, directions in which the curved portion of the internal channels curve, and/or extent to which the curved portion of the internal channels curves in a particular direction, may, in some embodiments, be modified to provide a desired radiation source arrangement to accommodate a particular anatomy and/or brachytherapy application. The curvature characteristics of the outer channels may be modified and adapted to accommodate the flexibility of wires or other transport means utilized to move a radiation source within the applicator 10, 100, 200 and the limitations thereof with respect to smoothly moving along a curved path.

Radiation sources that can be utilized in combination with the various applicator 10, 100, 200 embodiments disclosed herein include, by way of non-limiting example, iridium-192 (Ir-192), cesium-137 (Cs-137), or cobalt-60 (Co-60) seeds that, in some cases, may be carried by a flexible wire, catheter, needle, or other carrying device suitable for insertion into the internal channels of the applicator 10, 100, 200. In some embodiments and implementations, a radiation source loaded into the applicator 10, 100, 200 may be in the form of a train of radiation seeds. Loading techniques which may be employed to load a radiation source into the various embodiments of the applicators 10, 100, 200 disclosed herein are readily known within the art. The combination of an applicator 10, 100, 200 with a radiation source and/or device for carrying a radiation source can be characterized as an applicator assembly.

In some embodiments and implementations, a radiation source may be loaded into the various embodiments of the applicators 10, 100, 200 disclosed herein via an afterloading machine, which, in some embodiments and implementations, may be computer-controlled. In some embodiments and implementations, an image guidance system, such as a computed tomography (CT) system, magnetic resonance imaging (MRI) system, and/or ultrasound system may be employed to facilitate guidance of the applicators 10, 100, 200 to a desired location within an individual in need of brachytherapy. Treatment planning software can also be utilized in the treatment of an individual in need of brachytherapy to assist in the calculation of dose distribution and optimized source positioning for treatment of an individual.

Accordingly, the various applicator 10, 100, 200 embodiments disclosed herein can be utilized as part of a larger system that includes one or more of: an afterloading machine that assists in the sequential deposit of a radiation source into the internal channels of the applicator 10, 100, 200; an image guidance system; and/or treatment planning software stored in a computer-readable medium and executed by one or more processors.

The various components of the applicator 10, 100, 200 embodiments disclosed herein are preferably constructed of medical-grade materials. In this regard, and in some embodiments, the materials utilized to construct the components of the various applicator 10, 100, 200 embodiments comply with International Organization Standardization (ISO) 10993. In various embodiments, the elongated body 20, 120, 220 may be constructed, in whole or in part, of a plastic material, such as, by way of non-limiting example, acetal copolymer (POM-C), acrylic (PMMA), polycarbonate, polyether ether ketone (PEEK), polypropylene (PP), polyphenylsulfone (PPSU), ultra-high molecular weight polyethylene, polytetrafluoroethylene (PTFE), polysulfone (PSU), polyphenylene sulfide (PPS), polyvinylidene fluoride (PVDF), polyetherimide (PEI), polyphenylene oxide (PPO), derlin, plastics from FormLabs® BioMed series of compatible plastics, and combinations thereof. In some embodiments, the conduits 32, 34, 35, 36, 38 are constructed, in whole or in part, of a metal material, such as, by way of non-limiting example, titanium, stainless steel, cobalt-chromium, tantalum, platinum, magnesium, and combinations thereof. In some embodiments, some or all the components of the applicator 10, 100, 200 may be constructed, in whole or in part, of a radiopaque material or combination of radiopaque materials. In some embodiments, the portions of the elongated body 20, 120, 220 defining the internal channels 22, 24, 25, 26, 28, 122, 124, 125, 126, 128, 222, 224, 225, 226, 228 are constructed of a metal material, such as titanium, while the remaining portions of the elongated body 20, 120, 220 of the applicator 10, 100, 200 are constructed of a plastic material, such as PMMA.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. An intracavitary applicator for radiation oncology, comprising:
an elongated body configured to be inserted into a vaginal canal of an individual, the elongated body including
a proximal end,
a distal end, and
a plurality of internal channels configured to receive a radiation source and guide the radiation source from the proximal end toward the distal end within the elongated body, with the plurality of internal channels including multiple internal channels that include a curved portion and are spaced from a longitudinal axis of the elongated body, and without the plurality of internal channels converging toward a common point in the elongated body.

2. The intracavitary applicator of claim 1, wherein the curved portion of each of the multiple internal channels spaced from the longitudinal axis of the elongated body curves toward another adjacently positioned internal channel that is spaced from the longitudinal axis of the elongated body.

3. The intracavitary applicator of claim 1, wherein the curved portion of each of the multiple internal channels spaced from the longitudinal axis reduces a distance between the internal channel and the longitudinal axis.

4. The intracavitary applicator of claim 1, wherein the curved portion of one or more internal channels of the multiple internal channels spaced from the longitudinal axis curves in multiple directions.

5. The intracavitary applicator of claim 1, wherein the curved portion of each internal channel of the multiple channels spaced from the longitudinal axis is such that each internal channel of the multiple channels spaced from the longitudinal axis cannot be bisected by a single vertical plane.

6. The intracavitary applicator of claim 1, wherein no two internal channels of the multiple internal channels spaced from the longitudinal axis of the elongated body curve toward each other.

7. The intracavitary applicator of claim 1, wherein the plurality of internal channels includes a central channel that extends along the longitudinal axis.

8. The intracavitary applicator of claim 1, wherein each internal channel of the plurality of internal channels can be bisected by a single vertical plane.

9. The intracavitary applicator of claim 1, wherein the elongated body includes a cylindrical portion and a dome portion, the cylindrical portion defining the proximal end of the elongated body, and the dome portion defining the distal end of the elongated body.

10. The intracavitary applicator of claim 9, wherein each internal channel of the plurality of internal channels extends from the proximal end of the elongated body to a terminal point located in the dome portion of the elongated body.

11. The intracavitary applicator of claim 1, wherein the elongated body has a length ranging from about 6 cm to about 10 cm.

12. The intracavitary applicator of claim 1, wherein the elongated body has an outer diameter ranging from about 2.5 cm to about 3.5 cm.

13. The intracavitary applicator of claim 1, wherein the elongated body has an outer diameter of less than 3.5 cm.

14. The intracavitary applicator of claim 1, and further comprising: a plurality of conduits attached to the elongated body, such that each conduit of the plurality of conduits is in fluid communication with a internal channel of the plurality of internal channels.

15. The intracavitary applicator of claim 14, wherein the intracavitary applicator has a length of about 25 cm.

16. An intracavitary applicator for radiation oncology, comprising:
an elongated body configured to be inserted into a vaginal canal of an individual, the elongated body including
a cylindrical portion that defines a proximal end of the elongated body,
a dome portion that defines a distal end of the elongated body, and
a plurality of internal channels configured to receive a radiation source and guide the radiation source from the proximal end toward the distal end within the elongated body, the plurality of internal channels including
a central channel extending along a longitudinal axis of the elongated body,
multiple outer channels spaced from the central channel, each outer channel including a curved portion that curves toward another adjacently positioned outer channel, without any two outer channels curving toward each other.

17. The intracavitary applicator of claim 16, wherein each outer channel of the multiple outer channels curves in multiple directions.

18. The intracavitary applicator of claim 16, wherein each internal channel of the plurality of internal channels can be bisected by a single vertical plane.

19. The intracavitary applicator of claim 16, wherein each internal channel of the plurality of internal channels has a proximal open end located at the proximal end of the elongated body and a closed distal end located in the dome portion of the elongated body, and wherein the multiple outer channels do not converge toward the central channel.

20. The intracavitary applicator of claim 16, wherein each channel of the multiple outer channels cannot be bisected by a single vertical plane.
